(19) United States
(12) Patent Application Publication
(10) Pub. No.: US 2020/0281753 A1
(43) Pub. Date: Sep. 10, 2020

(54) Posture Correcting Device

(71) Applicant: Liling YU, Ningbo (CN)

(72) Inventor: Liling YU, Ningbo (CN)

(21) Appl. No.: 16/809,569

(22) Filed: Mar. 5, 2020

Related U.S. Application Data
(60) Provisional application No. 62/814,259, filed on Mar. 5, 2019.

(51) Int. Cl. A61F 5/05; A61F 5/055

(52) U.S. Cl. CPC A61F 5/05; A61F 5/055

(57) ABSTRACT

A posture correcting device includes a supporting body, wherein one end of the supporting body is disposed with a front supporting portion, wherein the supporting body is surrounded along one of the ends to form a wearing cavity, wherein an opening is formed between two ends of the supporting body.

### CROSS REFERENCE OF RELATED APPLICATION

This application is a non-provisional application that claims the benefit of priority under 35U.S.C. § 120 to a provisional application, application No. 62/814,259, filed Mar. 5, 2019. The afore-mentioned provisional application is hereby incorporated by reference in its entirety.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to a supporting device, and more particularly to a posture correcting device.

### Description of Related Arts

These days, many pillows focus on the support of the neck, paying attention to provide supporting force for the neck when a user is resting, especially during a nap or the like, so that the user can have the neck being stably supported while sleeping. When the user wears the pillow, it can well protect and support the head when the head is tilted back. But when the head is tilted forward, the force of the head is likely to cause the pillow to fall off from the neck, which does not provide a stable supporting force for the head.

It will be understood that, during sleeping, the user is stay in stationary and the head does not provide much pressure in most situations. It is very important to stay still and comfortable while in sleep. It is not only to maintain the support of the neck during sleeping, but also to keep corrections of the entire walking posture, especially the head posture, during walking.

Nowadays, many people are companied with electronic devices such as mobile phones and tablet PCs, so that during walking, especially when people using mobile phones, the entire head will goes down to watch screens. According to research, a person's head weighs about 5 kilograms. When the head is tilted forward to look at electronic devices such as mobile phones, it usually has an angle of 60° of the neck. Due to gravity and leverage, the neck muscles must bear more than 25 kilograms. After a long time, if the pressure on the head is too large, it will lead to hunchback, cervical spondylosis, etc., which will have a certain adverse impact on people's health.

In addition, many office white-collar workers now work with computers. In most cases, they need to keep their eyes and face to the computers. This requires people to rise the entire head. After a long time, the neck muscle will be rigidity, and the natural state will easily lead the head to fall forward.

The conventional neck pillow can solve the problem during resting, but can not solve these problems of falling head forward. The supporting force is mainly provided by the material inside the neck pillow, and a gap is formed at the lower jaw. While using the neck pillow, the head can have well supported at back and side, but lost supporting forward. In other words, when the neck pillow is turned and used under the lower jaw, the neck pillow is easily to fall off from the neck.

In addition, some people who are pursuing fashion and like to use headphones and large earrings, so that when the neck pillow is worn on the neck, the neck pillow is easy to resist on the headphones or earrings, which adversely affects the whole wearing effect.

### SUMMARY OF THE PRESENT INVENTION

The invention is advantageous in that it provides a posture correcting device, wherein when the posture correcting device is worn around the neck of a user, a supporting force is provided for the jaw, so that the user's lower jaw is capable of being maintained a normal state, and the entire head posture is corrected.

Another advantage of the invention is to provide a posture correcting device, wherein the posture correcting device is capable of being worn not only during walking but also during a resting state, and provides the user with supporting to the lower jaw during walking, avoiding tilting the head forward.

Another advantage of the invention is to provide a posture correcting device, wherein the posture correcting device fits the lower jaw of the user and is capable of being worn on the neck so that the user's head tilting degree is maintained in a normal range, and the user still can turn the head to left or right.

Another advantage of the invention is to provide a posture correcting device, wherein the posture correcting device can not only provide supporting force for the jaw when worn around the neck, and is also capable of supporting the cheeks.

Another advantage of the invention is to provide a posture correcting device, wherein the posture correcting device not only provides support force for the cheeks and jaw, and the posture correcting device is bound off at side thereof, which is distributed the force so that the pressure from the entire head is capable of being evenly distributed to all levels.

Another advantage of the invention is to provide a posture correcting device, wherein the side curve of the posture correcting device is designed in upper curve shape, simulating the feeling of the human hand holding the jaw so that the user can comfortably wear the posture correcting device.

Another advantage of the invention is to provide a posture correcting device, wherein both the upper and lower portions of the posture correcting device is capable of being symmetrically disposed so that the posture correcting device is capable of being used in a upside down manner to perform this operation.

Another advantage of the invention is to provide a posture correcting device, wherein the upper and lower portions of the posture correcting device are provided with a rounded curve for fitting the curves of the shoulders so that the user can comfortably wear the posture correcting device.

Another advantage of the invention is to provide a posture correcting device, wherein the posture correcting device is provided with a back curve and a chest curve so that the posture correcting device fits the front chest and the back of the user during wearing.

Another advantage of the invention is to provide a posture correcting device, wherein the posture correcting device is provided with a high and low mountain-type curve, and is provided with an ear-wearing cavity for the user's ear while providing a large supporting force so that the user can wear an extra device such as a headset.

Another advantage of the invention is to provide a posture correcting device, wherein the posture correcting device is provided with a double-peaks curve to provide a full range of support for the head and neck while providing a large supporting force.

Another advantage of the invention is to provide a posture correcting device wherein the posture correcting device is provided with a pulling device such that the posture correcting device is capable of being tightened by the pulling device to reduce the storage space.

Another advantage of the invention is to provide a posture correcting device wherein the posture correcting device is capable of being detached such that parts of the posture correcting device is capable of being used for other body parts.

Another advantage of the invention is to provide a posture correcting device, wherein the thickness of the posture correcting device is adjustable, and by adjusting the thickness of the posture correcting device, it is possible to serve for different neck heights and different comfort levels according to the user.

Another advantage of the invention is to provide a posture correcting device, wherein the posture correcting device can adjust the supporting position between the posture correcting device and the user by moving back and forward to serve for different neck heights and different comfort levels according to the user.

Another advantage of the invention is to provide a posture correcting device, wherein the posture correcting device comprises a supporting body and a coating, and the coating is provided with a connecting component, an adjusting component, a receiving component, and the like, which further assisting the supporting body to complete the wearing operation and other operations.

Additional advantages and features of the invention will become apparent from the description which follows, and may be realized by means of the instrumentalities and combinations particular point out in the appended claims.

According to the present invention, the foregoing and other objects and advantages are attained by a posture correcting device, comprising:

a supporting body, wherein one end of the supporting body is disposed with a front supporting portion, wherein the supporting body is surrounded along one of the ends to form a wearing cavity, wherein an opening is formed between two ends of the supporting body.

According to the present invention, the posture correcting device further comprises a coating for fitting the supporting body therein such that the posture correcting device can cover the supporting body.

According to the present invention, the front supporting portion is located in front of the supporting body, and the opening is positioned on the side of the supporting body.

According to the present invention, the front supporting portion further has a top surface and a bottom surface, wherein the top surface is an upper inclined surface and the bottom surface is a lower inclined surface.

According to the present invention, the supporting body further comprises a rear supporting portion and two side supporting portions, wherein the side supporting portions are disposed at two ends of the rear supporting portion, wherein the front supporting portion and the rear supporting portion is integrally formed by one of the side supporting portions.

According to the present invention, the supporting body further has a rear supporting portion and two side supporting portions, wherein the side supporting portions are disposed at two ends of the rear supporting portion, wherein the front supporting portion is detachably connected with the other side supporting portion.

According to the present invention, the supporting body further comprises a connecting component disposed between the front supporting portion and the other of the side supporting portion.

According to the present invention, the connecting component is provided as at least two connecting buckles, wherein the connecting buckles are disposed on the front supporting portion and the side supporting portion respectively.

According to the present invention, a upper concave surface is further formed in the top surface of the front supporting portion, wherein the upper concave surface is adapted for the curve of the lower jaw of the user during wearing.

According to the present invention, the front supporting portion and the side supporting portion form a distributing force receiving area, and the force points of the distributed force receiving area are evenly distributed.

According to the present invention, the side supporting portion has a top surface and a bottom surface, wherein a upper concave cavity is formed on the top surface, wherein a lower concave cavity is formed on the bottom surface, wherein the height of the side supporting portion is constantly vertically increasing from the middle to the sides.

According to the present invention, the binding component comprises two binding belts and a connecting buckle, wherein the binding belts are respectively provided at ends of the supporting body, wherein the connecting buckle is set on one of the binding belts, and the binding belt is connected with the other binding belt.

According to the present invention, the supporting body further has two side supporting portions, wherein the side supporting portions are disposed on sides of the front supporting portion, and a rear opening is formed between the side supporting portions.

According to the present invention, the binding component comprises two binding belts and a connecting buckle, wherein the binding belts are respectively provided at ends of the supporting body, wherein the connecting buckle is set on one of the binding belts, and the binding belt is connected with the other binding belt.

According to the present invention, the posture correcting device further comprises an adjusting component provided at the front supporting portion, wherein the front supporting portion adjusts height in a vertical direction by the adjusting component.

According to the present invention, the adjusting component comprises a adjustable belt and a stopper, wherein one end of the adjustable belt is fixed to one side of a vertical direction of the front supporting portion, and the adjustable belt is extended to the other side of the front supporting portion, and the adjustable belt is capable of being fixed in different positions through the stopper to drive front supporting portion to complete telescopic adjustment in the vertical direction.

According to the present invention, the adjusting component comprises at least one inflatable pillar and an inflating device, wherein the inflatable pillar is vertically built in the front supporting portion, and the inflating device is capable of adjusting the height of the inflatable pillar in the vertical direction to adjust the height of the front supporting portion in the vertical direction.

According to the present invention, wherein the inflatable device is a hand-pressure inflating device disposed outside the supporting body to communicate with the inflatable pillar.

According to the present invention, the posture correcting device further comprises a receiving component, wherein the receiving component comprises a tightening ring, wherein one end of the tightening ring is fixed to the outer side of the supporting body, and the tightening ring is elastic, wherein when the end of the supporting body is inwardly surrounded, the tightening ring is capable of being sleeved outside the supporting body.

According to the present invention, the receiving component of the posture correcting device comprises a locker and a tightening string, and the locker and one end of the tightening string are fixedly disposed on the outer side of the supporting body, and the tightening ring is elastic, wherein the locker is capable of being fixedly connected to the other end of the tightening string, wherein when the end of the supporting body is inwardly surrounded, the tightening ring and the locker are capable of being sleeved outside the supporting body.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of the supporting body according to a first preferred embodiment of the present invention.

FIG. 2A is a top view of the supporting body according to a first preferred embodiment of the present invention.

FIG. 2B is a front view of the supporting body according to a first preferred embodiment of the present invention.

FIG. 2C is a side view of the supporting body according to a first preferred embodiment of the present invention.

FIG. 2D is a perspective view according to a first preferred embodiment of the present invention.

FIG. 3 is a perspective view according to a first preferred embodiment of the present invention during wearing.

FIG. 4 is a top view according to a second preferred embodiment of the present invention.

FIG. 5 is a perspective view according to a second preferred embodiment of the present invention during wearing.

FIG. 6 is a perspective view according to a third preferred embodiment of the present invention during wearing.

FIG. 7 is a perspective view according to a fourth preferred embodiment of the present invention during wearing.

FIG. 8 is a top view according to a fifth preferred embodiment of the present invention.

FIG. 9 is a perspective view according to a sixth preferred embodiment of the present invention.

FIG. 10 is a perspective view an alternative mode of a sixth preferred embodiment of the present invention during wearing.

FIG. 11 is a perspective view an alternative mode of a sixth preferred embodiment of the present invention during wearing.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

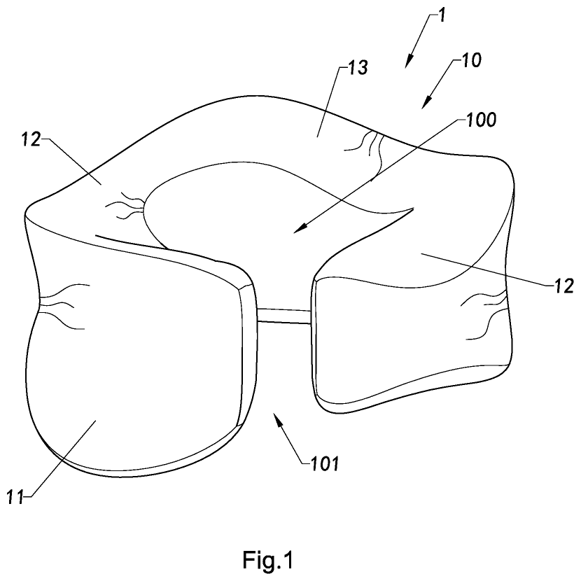

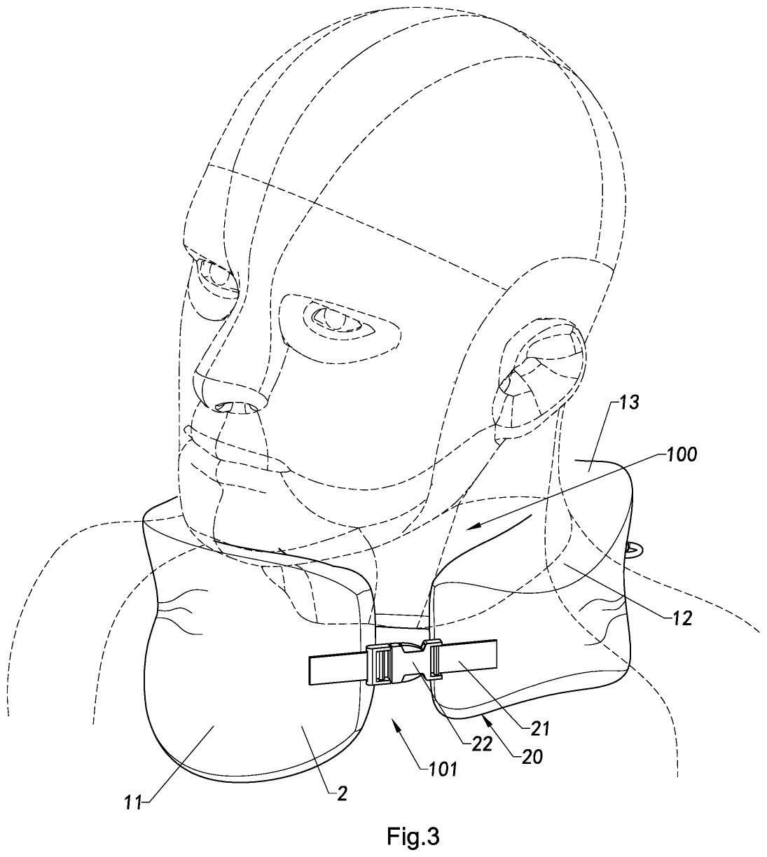

As shown in FIGS. 1, 2A-2C and 3, the present invention provides a first embodiment, wherein the first embodiment provides a posture correcting device 1, wherein the posture correcting device 1 is used for supporting a user's lower jaw. When the posture correcting device 1 is worn around the neck of the user, the posture correcting device 1 will provide a supporting force to the user's lower jaw to prevent the user's head from tilting forward. In other words, the posture correcting device 1 is for correcting the posture of the head of the user during walking.

It will be understood that the posture correcting device 1 is not simply filling the gaps between the lower jaw and the chest of the user, but provides a stable and safe supporting force for the user's lower jaw and even the entire head in such a space. When the user's lower jaw is lifted, the posture of the entire head will be well corrected.

Specifically, the posture correcting device 1 comprises a supporting body 10, and the supporting body 10 has two ends, such that when the supporting body 10 is surrounded with the opposite ends, a space 100 is formed in the interior of the supporting body 10. An opening 101 is formed between the two ends of the supporting body 10. It will be understood that the opening 101 is located at the side of the supporting body 10.

Different from the conventional symmetrical neck pillow, for example the opening in the middle, which is just under the jaw of the user. When the opening is in the middle, the conventional neck pillow can not provide stable supporting force to the lower jaw. Therefore, in the present invention, the opening 101 of the supporting body 10 is located at the side.

The positional relationship of the supporting body 10 is determined according to the position of the head corresponding to the user after being worn. According to the position of the user's head, it is divided into a front portion and a rear portion, wherein the front portion is further divided into a lower jaw portion and a cheek portion, that is to say the supporting body 10 correspondingly supports the lower jaw portion, the cheek portion and the rear portion. Thus, the supporting body 10 is divided into a front supporting portion 11, a side supporting portion 12 and a rear supporting portion 13, which in the present embodiment, the front supporting portion 11, the side supporting portion 12 and the rear supporting portion 13 are integrally formed.

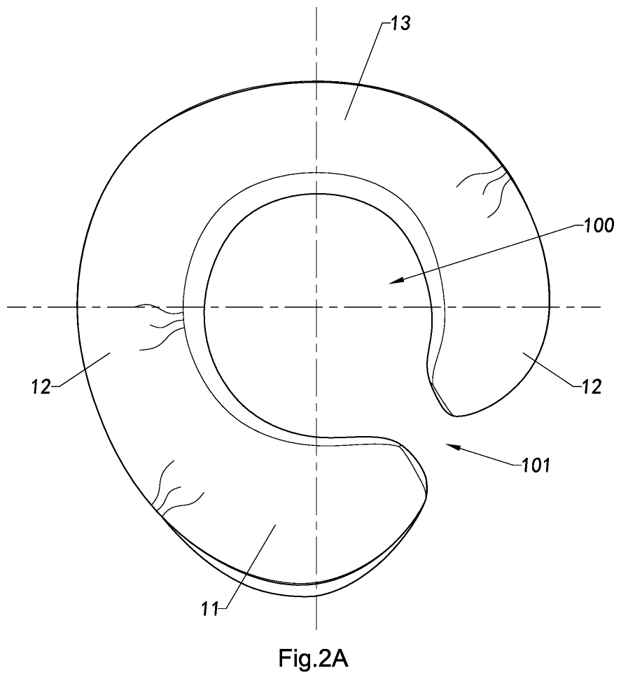

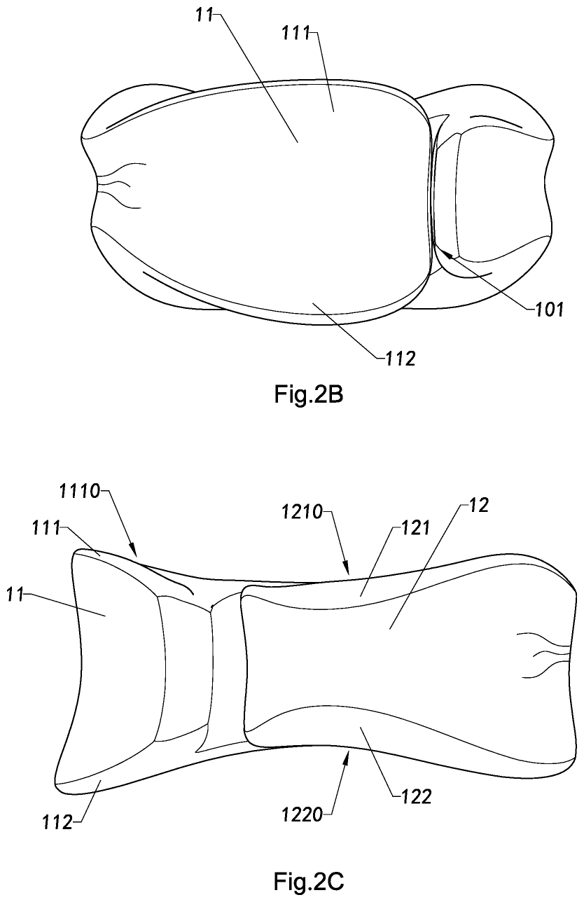

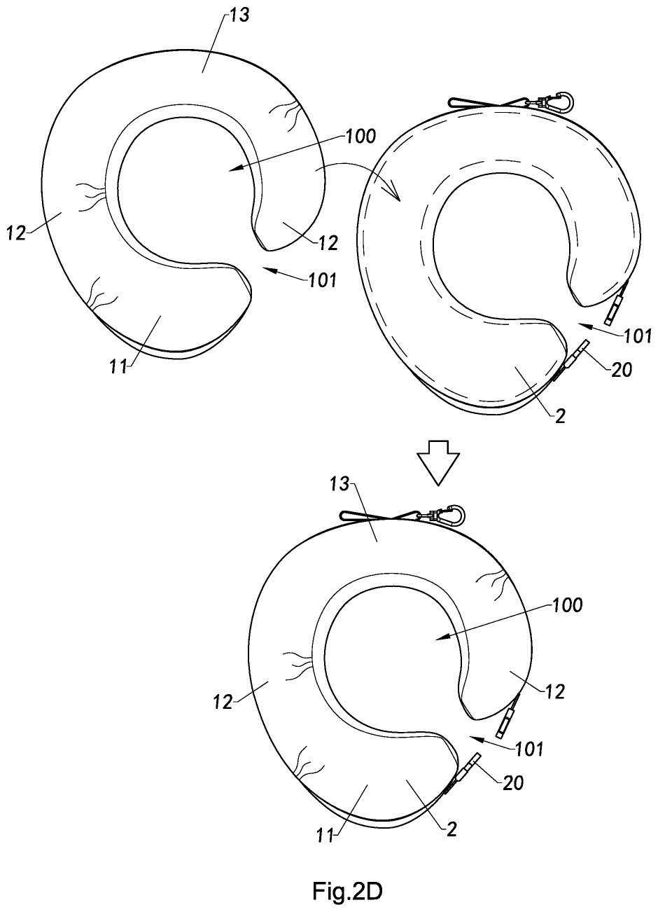

Preferably, as shown in FIG. 2A, a top view of the supporting body 10 is illustrated, wherein the position of the opening 101 is located in the middle of one of the side supporting portion 12. In other words, with the supporting body 10 in a Cartesian coordinate system, the front supporting portion 11 and the rear supporting portion 13 are in a first axial direction in cross section, and the opening 101 is disposed at 45° in the axial direction of the front supporting portion 11. It should be noted that the position of the opening 101 may be any position other than the front supporting portion 11.

When the supporting body 10 is worn around the neck of the user, the front supporting portion 11 is located directly in front of the head, and the side supporting portion 12 is located between the front of the head and the ears. The rear supporting portion 13 is located directly behind the head. It should be noted that the opening 101 is located in the side supporting portion 12. Therefore, the supporting body 10 has two of the side supporting portions 12 which on the left side and right side, and the opening 101 is located on one of the side supporting portions 12.

Furthermore, the front supporting portion 11 has a top surface 111 and a bottom surface 112, and the distance between the top surface 111 and the bottom surface 112. That is the thickness of the front supporting portion 11, is approximately equal to the general normal height of the user's jaw to the front chest. Thus, when the front supporting portion 11 is worn, the bottom surface 112 is against the user's chest portion, and the top surface 111 is against the user's lower jaw position.

Preferably, the top surface 111 is for supporting the lower jaw of a user. In particular, the top surface 111 is provided as an upper inclined surface 111, wherein the top surface 111 is extended upwardly from the inside to the outside in the transverse direction, such that the user's lower jaw is capable of being stabilized by being placed on the upper inclined surface 111. On the other hand, the bottom surface 112 is provided as a bottom inclined surface 112, wherein the bottom inclined surface 112 is extended downwardly from the inside to the outside in the transverse direction, so that the bottom inclined surface 112 fit the chest the of user.

The length of the bottom surface 112 in the lateral direction is larger than the top surface 111, so that the entire supporting effect is formed in a triangle type, for providing a stable supporting effect on the user's lower jaw. The bottom surface 111 is an inclined surface, which pressed against the chest of the user, and the angle of inclination of the inclined surface is to fit the user's chest.

When the lower jaw of the user touches on the top surface 111, the bottom surface 112 is pressed against the front chest of the user, and provides a supporting force for the lower jaw to support the lower jaw to prevent the lower jaw from moving downwardly which is to prevent the entire head from tilting forward. Thus, in normal wear, both the bottom surface 112 and the top surface 111 are completely pressed against the lower jaw and the front chest.

It should be noted that when the supporting body 10 is worn around the neck of the user, the front supporting portion 11 is snugly fixed at a position between the lower jaw and the front chest of the user. In this case, the supporting body 10 allows a user moving side to side. That is, the user may need to temporarily leave the jaw off the top surface 111 of the front supporting portion 11. Thus, the top surface 111 has an upper concave cavity 1110 for placing a user's lower jaw. On the one hand, providing a comfortable support for the user's lower jaw. On the other hand, when the user's head is rotated, the lower jaw can slide out of the upper concave cavity 1110, and when the user's head is turned back to the original position, the lower jaw can return to the upper concave cavity 1110 and then back to the normal wearing state. Meanwhile, during wearing the supporting body 10 around the user's head, when the user shakes the head, the supporting body 10 remains in the original position, so that when the head of the user is returning to the normal position, the supporting body 10 still against the lower jaw and the front chest.

The front supporting portion 11 is hold at top and bottom position by the user's lower jaw and chest. In other words, the top and bottom surfaces of the front supporting portion 11 are against on the lower jaw and the chest respectively. The front supporting portion 11 completes the support of the lower jaw at the top and bottom position.

On the other hand, the left and right positions of the front supporting portion 11 are respectively fixed by the side supporting portions 12. It will be understood that the side supporting portions 12 are located on sides of the front supporting portion 11, and the side supporting portions 12 form a height difference with the front supporting portion 11 in the lateral direction. That is, the side supporting portion 12 rises from the front supporting portion 11. Since the cheek and the cheek of the human body have a certain curve, the side supporting portion 12 and the front supporting portion 11 are formed in an undulating shape. By the undulating shape of the side supporting portion 12 and the front supporting portion 11, the cheek portion and the lower jaw are capable of being closer to each other.

In particular, the side supporting portion 12 has a top surface 121 and a bottom surface 122. During wearing, the top surface 121 of the side supporting portion 12 contacts the cheeks, and he bottom surface 122 is in contact with the shoulder, so the side supporting portion 12 is located between the shoulder and the cheek to provide support for the cheek. Moreover, the bottom surface 122 is formed in undulating curve, and the top surface 121 is formed in undulating curve. While the side supporting portion 12 supports the cheek portion, the front supporting portion 11 supports the lower jaw.

The top surface 121 of the side supporting portion 12 is formed an upper concave cavity 1210. When the user has worn the headphone, the upper concave cavity 1210 is to provide a headset placement space, so that the supporting force is stable while using the headphone.

A lower concave cavity 1220 is formed on the bottom surface 122 of the side supporting portion 12. When the user wears the posture correcting device 1, the lower concave cavity 1220 fits the curve of the user's shoulder, which is convenient and stable on the one hand. On the other hand, the pressure applied to the shoulder is distributed by the posture correcting device 1.

After worn, the side supporting portion 12 is completed to support the cheeks, and further fix the position of the front supporting portion 11 such that the position of the front supporting portion 11 is always in the front.

Furthermore, the rear supporting portion 13 has a semicircular shape, and the rear supporting portion 13 forms a supporting space for supporting a user's rear neck. The rear supporting portion 13 has a top surface 131 and a bottom surface 132. After being worn, the bottom surface 132 fits over the back to the shoulder of the user and, and the top surface 131 providing a support surface for the user to recline.

It is worth to mention that after the front supporting portion 11 and the side supporting portion 12 are fixed, the rear supporting portion 13 is fixed at the rear and provides pulling force on the front supporting portion 11 and the side supporting portion 1. Therefore, it will be understood that the front supporting portion 11, the side supporting portion 12 and the rear supporting portion 13 have a fixed position when wearing. Therefore, when the user wears the posture correcting device 1, relative positional relationship between the posture correcting device 1 and the head portion of the user is determined. If there is a large change in the relative positions, both can not be matched with each other.

Therefore, it will be understood that during wearing, the front supporting portion 11 corresponds to the lower jaw of the human body, so the front supporting portion 11 can support the lower jaw of the human body so that the user's head remains upright to avoid some injuries caused by neck bending.

In addition, the side supporting portion 12 and the front supporting portion 11 form a distributing force receiving area, and the force receiving points of the distributing force receiving area are relatively distributed, so that the whole part is relatively evenly stressed. Therefore, the force applied to the various parts of the user's head during wearing is similar the same.

As shown in FIG. 2D, the present invention provides a coating 2 covered the supporting body 10, and the coating 2 is capable of supporting the package body 10 entirely, so that the supporting body 10 is protected. Furthermore, the inflatable pillow device further comprises a binding component 20, wherein the binding component 20 is disposed on the coating 2, wherein the binding component 20 comprises a binding belt 21 and a connecting buckle 22, wherein the binding belt 21 is disposed at side of the opening 101, and the connecting buckle 22 is disposed on the other side of the opening 102. That is the binding band 21 is disposed at one end of the front supporting portion 11, and the connecting buckle 22 is provided at one end of the side supporting portion 12.

It will be understood that the binding component 20 can also be other connection manners, such as a buckle, a button, etc., for further restraining the opening 101 during wearing, so that the wearing is more firm and stable.

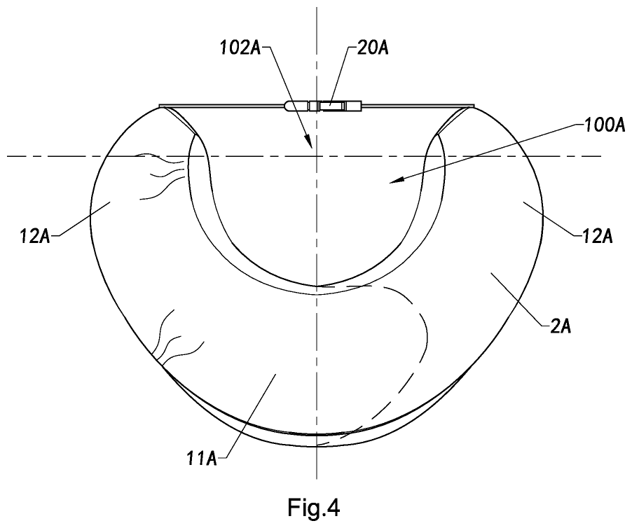

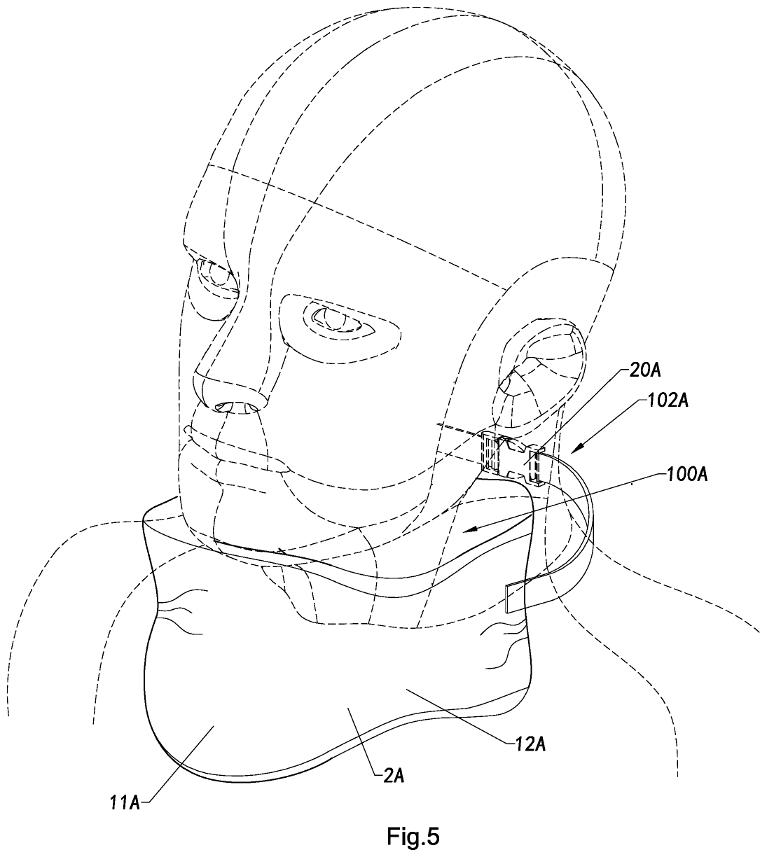

As shown in FIG. 4-5, the present invention provides a second embodiment. In the second embodiment, the present invention provides a posture correcting device 1A, wherein the posture correcting device 1A comprises a supporting body 10A, a coating 2A, and a binding component 20A, wherein the supporting body 10A is fitted in the coating 2A, so that the coating 2A is capable of covering the supporting body 10A, and the binding component 20A is disposed on the coating 2A, wherein the supporting body 10A comprises a front supporting portion 11A and two side supporting portions 12A located at the sides of the front supporting portion 11A.

Furthermore, the front supporting portion 11A has a top surface 111A and a bottom surface 112A, and the distance between the top surface 111A and the bottom surface 112A is the thickness of the front supporting portion 11A, and is approximately equal to the general normal height of the user's jaw to the front chest. Therefore, when the front supporting portion 11A is worn, the bottom surface 112A is pressed against the front chest of the user, and the top surface 111A is pressed against the lower jaw of the user.

When the lower jaw of the user contacts the top surface 111A, the bottom surface 112A presses against the front chest of the user, and provides a supporting force for the lower jaw to support the lower jaw and prevent the lower jaw from moving down which is to prevent the entire head from leaning forward. Therefore, in the normal wearing, the bottom surface 112A and the top surface 111A are completely against the lower jaw and the front chest portion.

It is to be noted that when the supporting body 10A is worn around the neck of the user, the front supporting portion 11A is snugly fixed to a position between the lower jaw and the front chest of the user. In addition, the opening 101A is provided on the one side of the side supporting portion 12A. Therefore, the supporting body 10A allows the user to shake head to left and right, and the supporting body 10A remains in the original position during wearing and shaking the user's head to the left and right sides so that when the user's head returning to the normal position, the lower jaw and the front chest are still be against.

The front supporting portion 11A is clamped in the vertical position by the user's chest and the front jaw. In other words, the upper and lower surfaces of the front supporting portion 11A are respectively against the lower jaw and the front chest of the user.

On the other hand, the left and right positions of the front supporting portion 11A are respectively fixed by the side supporting portions 12A, and the side supporting portions 12A are provided on sides of the front supporting portion 11A.

After the posture correcting device 1A is worn, the front supporting portion 11A and the side supporting portion 12A correspond to the lower jaw and the cheek portion of the user, and the binding component 20A comprises a binding band 21A and a connecting buckle 22A. The binding band 21A is respectively disposed at the ends of the side supporting portions 12A. The connecting buckle 22A is provided on the one side of the binding band 21A, and the connecting buckle 22A connects on both sides of the side supporting portions 12A. The binding band 21A pulls the front supporting portion 11A and the side supporting portion 12A to be fixed at a position of the user's lower jaw and the cheek portion.

The position of the binding band 21A fastened with the connecting buckle 22A is capable of being adjusted to tighten or loosen the front supporting portion 11A and the side supporting portion 12A for supporting the user. On the other hand, the shape of the lower jaw and the cheek of the different users are slightly different, or the height of the lower jaw and the cheek is slightly different from the height of the neck and the front chest. Therefore, the front supporting portion 11A and the side supporting portion 12A are adjusted by the tightness of the binding band 21A to fit with the user's head.

It will be understood that the binding component 20A can also be other restraining manners, such as a buckle, a buckle, and the like.

It is worth to mention that, in the present embodiment, the side supporting portions 12A are respectively disposed at sides of the front supporting portion 11A, and an rear opening 102A is formed between the side supporting portions 12A, which is different from the first embodiment that the rear opening 102A is located on the rear. Therefore the present embodiment is only suitable for supporting the lower jaw of the user.

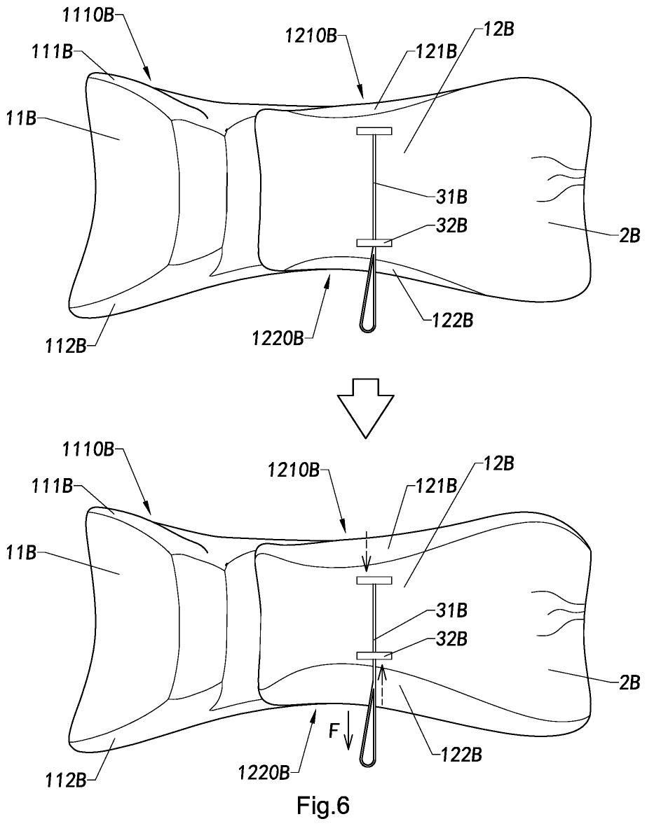

As shown in FIG. 6, the present invention provides a third embodiment. The third embodiment provides a posture correcting device 1B, wherein the posture correcting device 1B comprises a supporting body 10B, a coating 2B, and an adjusting component 30B, wherein the supporting body 10B is fitted inside the coating 2B such that the coating 2B is capable of covering the supporting body 10B, and the adjusting component 30B is disposed on the coating 2B. The supporting body 10B has two ends, and the supporting body 10B is surrounded so that the opposite ends are facing each other. A wearing cavity 100B is formed around inside the supporting body 10B and an opening 101B is formed between the ends of the supporting body 10B. It will be understood that the opening 101B of the supporting body 10B is located at the side.

The positional relationship of the supporting body 10B is determined according to the position of the head corresponding to the user after being worn. According to the position of the user's head, it is divided into a front portion and a rear portion, wherein the front portion is further divided into a lower jaw portion and a cheek portion, that is to say the supporting body 10B correspondingly supports the lower jaw portion, the cheek portion and the back portion. Thus, the supporting body 10B is divided into a front supporting portion 11B, a side supporting portion 12B and a rear supporting portion 13B, which in the present embodiment, the front supporting portion 11B, the side supporting portion 12B and the rear supporting portion 13B are integrally formed.

When the supporting body 10B is worn around the neck of the user, the front supporting portion 11B is located directly in front of the head, and the side supporting portion 12B is located between the front of the head and the ears. The rear supporting portion 13B is located directly behind the head. It should be noted that the opening 101B is located in the side supporting portion 12B. Therefore, the supporting body 10B has the side supporting portions 12B on the left or right sides, and the opening 101B is located on other side thereof.

Furthermore, the front supporting portion 11B has a top surface 111B and a bottom surface 112B, and the distance between the top surface 111B and the bottom surface 112B is the thickness of the front supporting portion 11B, and is approximately equal to the general normal height of the user's jaw to the front chest. Therefore, when the front supporting portion 11B is worn, the bottom surface 112B presses against the front chest portion of the user, and the top surface 111B presses the lower jaw of the user.

When the lower jaw of the user contacts the top surface 111B, the bottom surface 112B presses against the front chest of the user, and provides a supporting force for the lower jaw to support the lower jaw and prevent the lower jaw from moving down is to prevent the entire head from leaning forward. Therefore, in the normal wearing condition, the bottom surface 112B and the top surface 111B are completely against the lower jaw and the front chest portion.

It is to be noted that when the supporting body 10B is worn around the neck of the user, the front supporting portion 11B is fitted to the position between the lower jaw and the front chest of the user. In addition, the opening 101B is provided on the one of the side supporting portion 12B. Therefore, the supporting body 10B allows the user to shake head to left and right, and the supporting body 10B remains in the original position during wearing and shaking the user's head to the left and right sides so that when the user's head returning to the normal position, the lower jaw and the front chest are still be against.

The front supporting portion 11B is clamped in the vertical position by the user's chest and the front jaw. In other words, the upper and lower surfaces of the front supporting portion 11B are respectively against the lower jaw and the front chest of the user.

On the other hand, the left and right positions of the front supporting portion 11B are respectively fixed by the side supporting portions 12B, and the side supporting portions 12B are provided on sides of the front supporting portion 11B.

Preferably, wherein the position of the opening 101B is located in the middle of the side supporting portion 12B on one side thereof. That is the supporting body 10B is in a Cartesian coordinate system, the front supporting portion 11B and the rear supporting portion 13B are in a first axial direction in cross section, and the opening 101B is disposed at 45B ° in the axial direction of the front supporting portion 11B. It should be noted that the position of the opening 101B may be any position other than the front supporting portion 11B.

It will be understood that the side supporting portions 12B are located on both sides of the front supporting portion 11B, and the side supporting portions 12B forms a height difference with the front supporting portion 11B in the planar direction. That is the side supporting portion 12B rises from the front supporting portion 11B. Since the cheek and the cheek of the human body have a certain curve, the side supporting portion 12B and the front supporting portion 11B is formed as an undulating shape.

Specifically, the side supporting portion 12B has a top surface 121B and a bottom surface 122B. During wearing, the top surface 121B of the side supporting portion 12B contacts the cheeks, and the bottom surface 122B is in contact with the shoulder, so the side supporting portion 12B is located between the shoulder and the cheek to provide support for the cheek. Moreover, the bottom surface 122B is formed in undulating curve, and the top surface 121B is formed in undulating curve. While the side supporting portion 12B supports the cheek portion, the front supporting portion 11B supports the lower jaw.

After worn, the side supporting portion 12B is to complete to support cheeks while further securing the position of the front supporting portion 11B, such that the front supporting portion 11B is always in front.

Preferably, the rear supporting portion 13B has a semicircular shape, and the rear supporting portion 13B forms a supporting space for supporting a user's rear neck. The rear supporting portion 13B has a top surface 131B and a bottom surface 132B. After being worn, the bottom surface 132B fits over the back to the shoulder of the user and, and the top surface 131B providing a support surface for the user to recline.

It is worth to mention that after the front supporting portion 11B and the side supporting portion 12B are fixed, the rear supporting portion 13B is fixed at the rear and provides pulling force on the front supporting portion 11B and the side supporting portion 1B. Therefore, it will be understood that the front supporting portion 11B, the side supporting portion 12B and the rear supporting portion 13B have a fixed position when wearing. Therefore, when the user wears the posture correcting device 1B, relative positional relationship between the posture correcting device 1B and the head portion of the user is determined. If there is a large change in the relative positions, both can not be matched with each other.

The adjusting component 30B is disposed on the coating 2B to adjust the supporting body 10B, wherein the height of the upper and lower positions of the front supporting portion 11B is adjusted by the adjusting component 30B. The relative distance between the top surface 111B and the bottom surface 112B of the front supporting portion 11B is consistent in a normal state.

Specifically, the adjusting component 30B comprises a adjustable belt 31B and a stopper 32B, wherein one end of the adjustable belt 31B is disposed at an upper portion of the front supporting portion 11B, and the adjustable belt 31B is extended from the upper portion to the lower portion of the supporting front portion 11B out of the bottom surface 112B of the front supporting portion 11B. Furthermore, the stopper 32B is disposed in the bottom surface 112B, and the adjustable belt 31B passes through the stopper 32B, wherein the stopper 32B can prevent the adjustable belt 31B from moving, thus the adjustable belt 31B is capable of being pulled up to adjust the height of the front supporting portion 11B, And when determined, the stopper 32B is activated to block the adjustable belt 31B moves. The movement of the adjustable belt 31B causes the height of the front supporting portion 11B to be adjusted.

Therefore, according to the above embodiment, when the posture correcting device 1B is worn around the neck of the user, the overall height of the front supporting portion 11B is capable of being adjusted according to the adjustment of the adjustable belt 31B of the adjusting component 30B to be adapted for different users.

It will be understood that the positions of the adjustable belt 31B and the stopper 32B are capable of being changed. In another embodiment, one end of the stopper 31B disposed in a lower portion of the front portion supporting and the adjustable belt 31B is extended from lower portion of the front supporting portion 11B out of the top surface 112B of the front supporting portion 11B. The stopper 32B is disposed on the top surface 112B, and when the adjustable belt 31B through the stopper 32B, the stopper 32B can prevent the adjustable belt 31B moving.

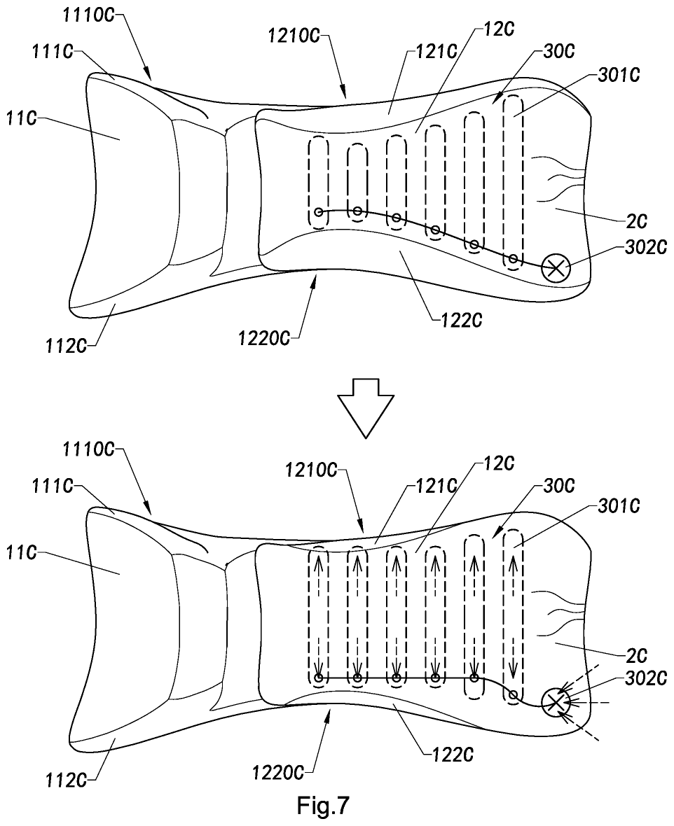

As shown in FIG. 7, the present invention provides a fourth embodiment. The fourth embodiment provides a posture correcting device 1C, wherein the posture correcting device 1C comprises a supporting body 10C, a coating 2C, and an adjusting component 30C, wherein the supporting body 10C is fitted inside the coating 2C such that the coating 2C is capable of covering the supporting body 10C, and the adjusting component 30C is disposed on the coating 2C. The supporting body 10C has two ends, and the supporting body 10C is surrounded so that the ends are facing each other. A wearing cavity 100C is formed around inside the supporting body 10C and an opening 101C is formed between the ends of the supporting body 10C. It will be understood that the opening 101C of the supporting body 10C is located at the side.

The positional relationship of the supporting body 10C is determined according to the position of the head corresponding to the user after being worn. According to the position of the user's head, it is divided into a front portion and a rear portion, wherein the front portion is further divided into a lower jaw portion and a cheek portion, that is to say the supporting body 10C correspondingly supports the lower jaw portion, the cheek portion and the back portion. Thus, the supporting body 10C is divided into a front supporting portion 11C, a side supporting portion 12C and a rear supporting portion 13B, which in the present embodiment, the front supporting portion 11C, the side supporting portion 12C and the rear supporting portion 13C are integrally formed.

When the supporting body 10C is worn around the neck of the user, the front supporting portion 11C is located directly in front of the head, and the side supporting portion 12C is located between the front of the head and the ears. The rear supporting portion 13C is located directly behind the head. It should be noted that the opening 101C is located in the side supporting portion 12C. Therefore, the supporting body 10C has the side supporting portions 12C on the left or right sides, and the opening 101C is located on other side thereof.

Furthermore, the front supporting portion 11C has a top surface 111C and a bottom surface 112C, and the distance between the top surface 111C and the bottom surface 112C is the thickness of the front supporting portion 11C, and is approximately equal to the general normal height of the user's jaw to the front chest. Therefore, when the front supporting portion 11C is worn, the bottom surface 112C presses against the front chest portion of the user, and the top surface 111C presses the lower jaw of the user.

When the lower jaw of the user contacts the top surface 111C, the bottom surface 112C presses against the front chest of the user, and provides a supporting force for the lower jaw to support the lower jaw and prevent the lower jaw from moving down is to prevent the entire head from leaning forward. Therefore, in the normal wearing condition, the bottom surface 112C and the top surface 111C are completely against the lower jaw and the front chest portion.

It is to be noted that when the supporting body 10C is worn around the neck of the user, the front supporting portion 11C is fitted to the position between the lower jaw and the front chest of the user. In addition, the opening 101C is provided on the one of the side supporting portion 12C. Therefore, the supporting body 10C allows the user to shake head to left and right, and the supporting body 10C remains in the original position during wearing and shaking the user's head to the left and right sides so that when the user's head returning to the normal position, the lower jaw and the front chest are still be against.

The front supporting portion 11C is clamped in the vertical position by the user's chest and the front jaw. In other words, the upper and lower surfaces of the front supporting portion 11C are respectively against the lower jaw and the front chest of the user.

On the other hand, the left and right positions of the front supporting portion 11C are respectively fixed by the side supporting portions 12C, and the side supporting portions 12C are provided on sides of the front supporting portion 11C.

Preferably, wherein the position of the opening 101C is located in the middle of the side supporting portion 12C on one side thereof. That is the supporting body 10C is in a Cartesian coordinate system, the front supporting portion 11C and the rear supporting portion 13C are in a first axial direction in cross section, and the opening 101C is disposed at 45° in the axial direction of the front supporting portion 11C. It should be noted that the position of the opening 101C may be any position other than the front supporting portion 11C.

It will be understood that the side supporting portions 12C are located on both sides of the front supporting portion 11C, and the side supporting portions 12C forms a height difference with the front supporting portion 11C in the planar direction. That is the side supporting portion 12C rises from the front supporting portion 11C. Since the cheek and the cheek of the human body have a certain curve, the side supporting portion 12C and the front supporting portion 11C is formed as an undulating shape.

Specifically, the side supporting portion 12C has a top surface 121C and a bottom surface 122C. During wearing, the top surface 121C of the side supporting portion 12C contacts the cheeks, and the bottom surface 122C is in contact with the shoulder, so the side supporting portion 12C is located between the shoulder and the cheek to provide support for the cheek. Moreover, the bottom surface 122C is formed in undulating curve, and the top surface 121C is formed in undulating curve. While the side supporting portion 12C supports the cheek portion, the front supporting portion 11C supports the lower jaw.

After worn, the side supporting portion 12C is to complete to support cheeks while further securing the position of the front supporting portion 11C, such that the front supporting portion 11C is always in front.

Preferably, the rear supporting portion 13C has a semicircular shape, and the rear supporting portion 13C forms a supporting space for supporting a user's rear neck. The rear supporting portion 13C has a top surface 131C and a bottom surface 132C. After being worn, the bottom surface 132C fits over the back to the shoulder of the user and, and the top surface 131C providing a support surface for the user to recline.

It is worth to mention that after the front supporting portion 11C and the side supporting portion 12C are fixed, the rear supporting portion 13C is fixed at the rear and provides pulling force on the front supporting portion 11C and the side supporting portion 1C. Therefore, it will be understood that the front supporting portion 11C, the side supporting portion 12C and the rear supporting portion 13C have a fixed position when wearing. Therefore, when the user wears the posture correcting device 1C, relative positional relationship between the posture correcting device 1C and the head portion of the user is determined. If there is a large change in the relative positions, both can not be matched with each other.

The adjusting component 30C is disposed on the coating 2C to adjust the supporting body 10C, wherein the height of the upper and lower positions of the front supporting portion 11C is adjusted by the adjusting component 30C. The relative distance between the top surface 111C and the bottom surface 112C of the front supporting portion 11C is consistent in a normal state.

In details, the adjusting component 30C comprises a plurality of inflatable pillars 301C and an inflating device 302C. The front supporting portion 11C is provided within the plurality of inflatable pillars 301C, and the inflating device 302C is capable of inflating and deflating the inflatable pillar 301C. Furthermore, by adjustment of the inflatable pillar 301C of the inflation and deflation, the height of the front supporting portion 301C is adjusted with the inflatable pillar 301C.

It is worth to mention that the inflating device 302C is provided a push-type inflating device 302C, the user can complete inflation and deflation of inflatable pillar 301C by simply pressing on the inflating device. Preferably, the inflatable pillar 301C is embedded in the front supporting portion 11C with the inflating device 302C disposed outside the pillow main body 10C. Therefore, the height of the front supporting portion 11C is adjusted by the adjusting component 30C to fit different users.

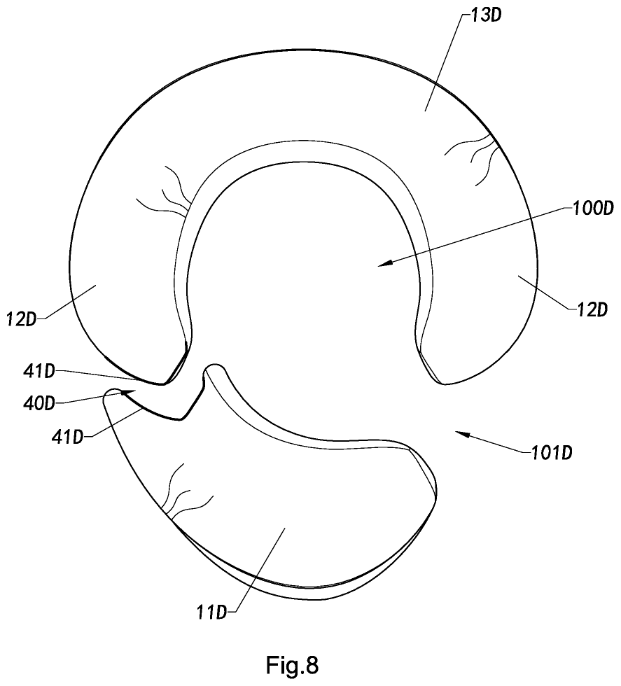

As shown in FIG. 8, the present invention provides a fifth embodiment. The fifth embodiment provides a posture correcting device 1D, wherein the posture correcting device 1D comprises a supporting body 10D and a connecting component 40D, which said supporting body 10D comprises a front supporting portion 11D, a side supporting portion 12D and a rear supporting portion 13D, wherein said front supporting portion 11D is connected with the side supporting portion 12D through the connecting component 40D.

When the front supporting portion 11D is separated from the side supporting portion 12D, the side supporting portion 12D and the rear supporting portion 13D may still provide support for the user.

Specifically, the connecting component 40D comprises a plurality of connecting buckles 41D, wherein the connecting buckles 41D are disposed at the seams between the side supporting portions 12D and the front supporting portion 11D. When the front supporting portion 11D and the side supporting portion 12D are in contact with each other, the connecting buckle 41D closely connects the front supporting portion 11D and the side supporting portion 12D.

In the present embodiment, the front supporting portion 10D is detachably connected with the side supporting portion 12D, wherein when the front supporting portion 10D and the side supporting portion 12D are connected, the function is similar as the first embodiment of the present invention. And when the front supporting portion 10D and the side supporting portion 12D are separated from each other, the side supporting portion 12D and the rear supporting portion 13D are shaped as a U-shaped pillow, and the front supporting portion 10D alone is capable of being used to provide support when the user is lying on the table.

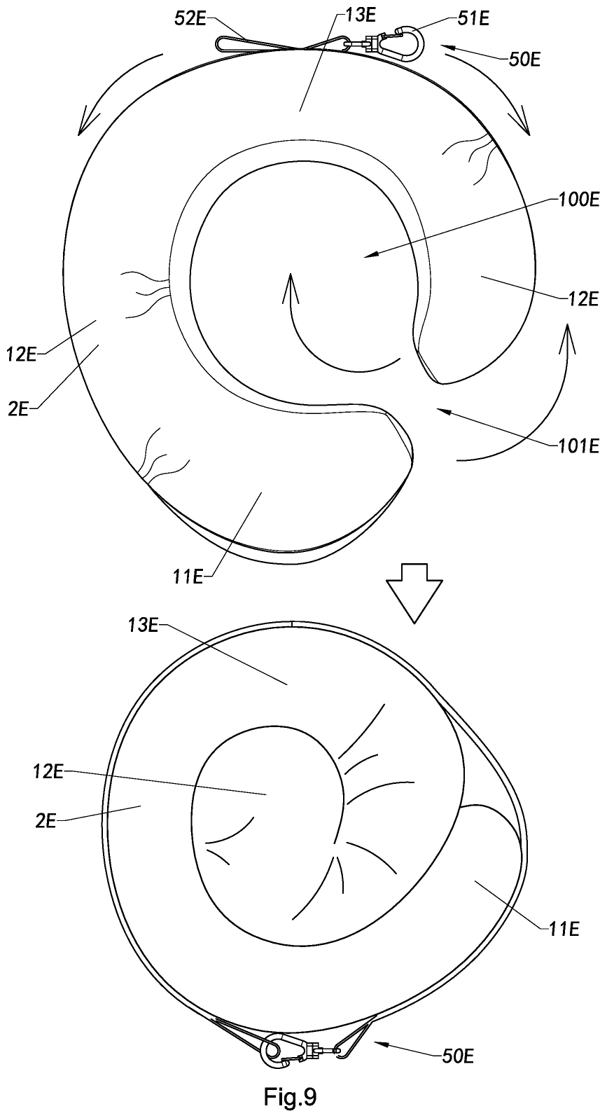

As shown in FIG. 9, the present invention further provides a sixth embodiment of a posture correcting device. The sixth embodiment provide a posture correcting device 1E, wherein the posture correcting device 1E comprises a supporting body 10E. The supporting body 10E comprises a front supporting portion 11E, a side supporting portion 12E and a rear supporting portion 13E, wherein the posture correcting device 1E has two ends, one end of which is extended from the other end and the supporting body 10E surrounds to form a wearing space 100E and an opening 101E.

In the posture correcting device 1E, one end of the posture correcting device 1E is provided with the front supporting portion 11E, and it will be understood that the front supporting portion 11E is in a large size, so when needed storage, the posture correcting device 1E is provided with one end of the front supporting portion 11E relatively fixed while the other end is tightened inward.

In an embodiment, the posture correcting device 1E further comprises a coating 2E covering the supporting body 1E. One end of the coating 2A is provided with a receiving component 50E, wherein said receiving component 50E comprises a locker 51E and a tightening string 52E, wherein one end of the tightening string 52E is disposed at any outside surface position of the posture correcting device 1E, wherein the locker 51E is disposed in one end of the posture correcting device 1E. Preferably, the tightening string 52E is disposed near to the locker 51E. When the other end of the posture correcting device 1E is inwardly rounded to reduced the overall volume until it cannot be contracted, the tightening string 52E is stretched to the locker 51E and the locker 51E is fixed with the tightening string 52E, and the tightening string 52E can bind the posture correcting device 1E to achieve the purpose of receiving the posture correcting device 1E.

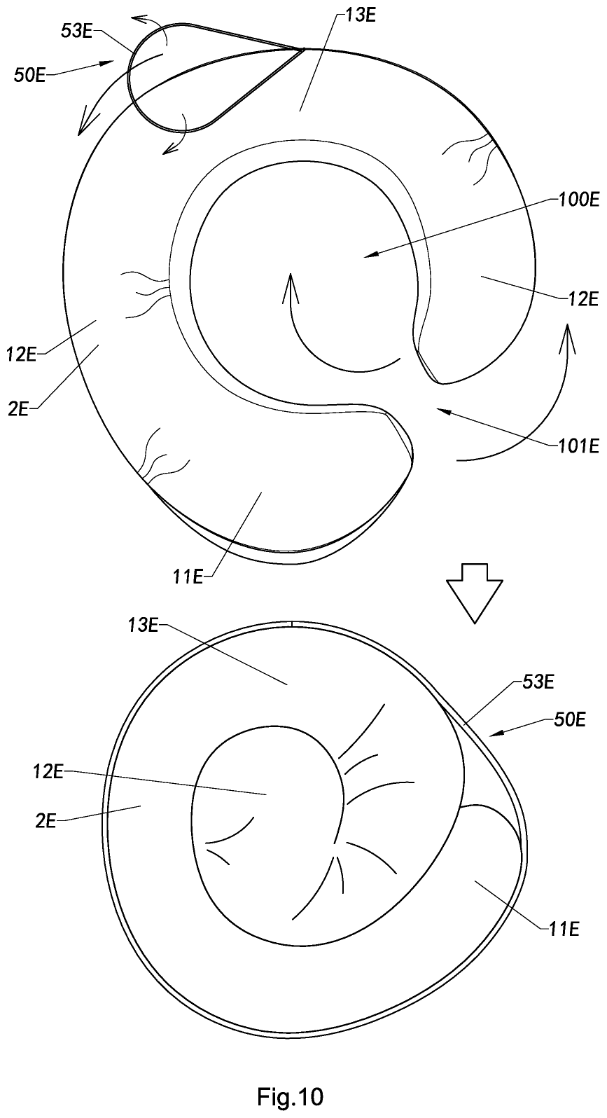

As shown in FIG. 10, the present embodiment provides an alternative embodiment, wherein the receiving component 50E comprises a tightening ring 53E, and the tightening ring 53E is capable of being stretched to form a pull-tab, and when the other end of the posture correcting device 1E surrounds and reduces the entire volume, the tightening ring 53E is capable of being sleeved on the outer side surface of the posture correcting device 1E.

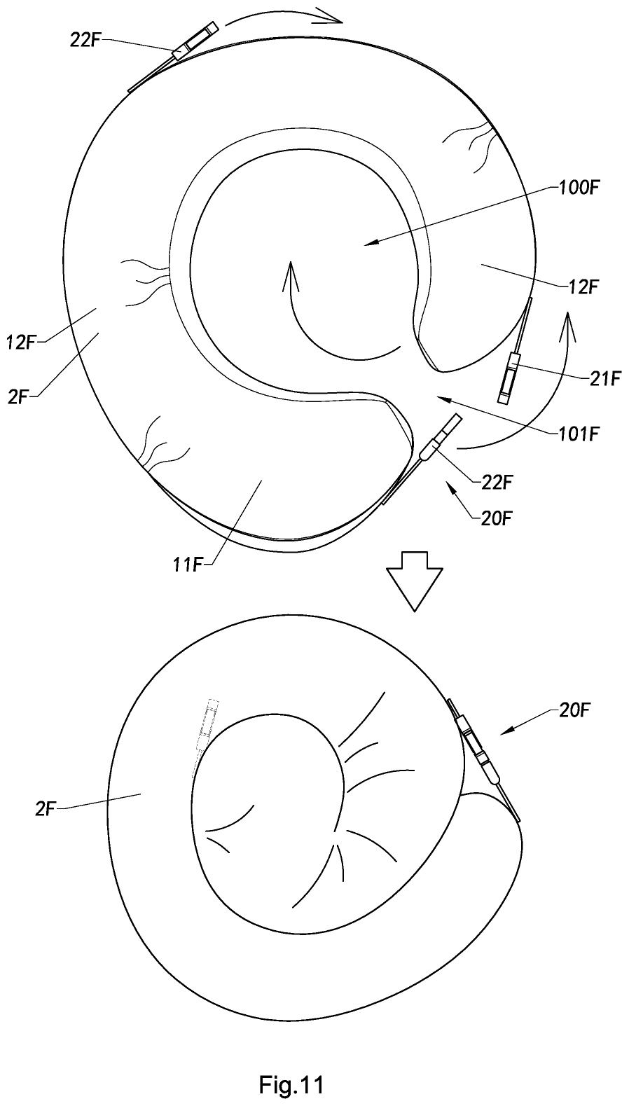

As shown in FIG. 11, the present invention provides another alternative embodiment, which provides a posture correcting device 1F, wherein the posture correcting device 1F comprises a supporting body 10F and a coating 2F, wherein the supporting body 10F is wrapped in the coating 2F so that the coating 2F can cover the supporting body 10F. One end of the coating 2F is provided with a connecting component 20F, wherein the connecting component 20F comprises a connecting buckle 21F and two connectors 22F, wherein one end of the connecting buckle 21F is fixed at one end of the posture correcting device 1F, and the supporting body 10F forms a wearing space 100F and an opening 101F. The connector 22F is disposed at the end of the opening 101 F, and the other connector 22F is disposed at the other end, and when the other end of the posture correcting device 1F surrounds and reduces the entire volume until it cannot be further contracted. The is capable of being pulled to fit over the outer surface of the posture correcting device 1F. The posture correcting device 1F enables to be accommodated by the above two manners, and the volume of the posture correcting device 1F is reduced to facilitate receiving and storage.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention comprises all modifications encompassed within the spirit and scope of the following claims.

What is claimed is:

1. A posture correcting device, comprising:
a supporting body, wherein one end of the supporting body is disposed with a front supporting portion, wherein the supporting body is surrounded along one of the ends to form a wearing cavity, wherein an opening is formed between two ends of the supporting body.

2. The posture correcting device as recited in claim 1, further comprising a coating fitting the supporting body therein such that the posture correcting device covers the supporting body.

3. The posture correcting device as recited in claim 2, further comprising a binding component disposed on the coating for connecting the coating covering the opening of the supporting body.

4. The posture correcting device as recited in claim 1, wherein the front supporting portion is located in front of the supporting body, and the opening is positioned on the side of the supporting body.

5. The posture correcting device as recited in claim 2, wherein the front supporting portion further has a top surface and a bottom surface, wherein the top surface is an upper inclined surface and the bottom surface is a lower inclined surface.

6. The posture correcting device as recited in claim 3, wherein the supporting body further comprises a rear supporting portion and two side supporting portions, wherein the side supporting portions are disposed at two ends of the rear supporting portion, wherein the front supporting portion and the rear supporting portion is integrally formed by one of the side supporting portions.

7. The posture correcting device as recited in claim 3, wherein the supporting body further has a rear supporting portion and two side supporting portions, wherein the side supporting portions are disposed at two ends of the rear supporting portion, wherein the front supporting portion is detachably connected with the other side supporting portion.

8. The posture correcting device as recited in claim 5, wherein the supporting body further comprises a connecting component disposed between the front supporting portion and the side supporting portion.

9. The posture correcting device as recited in claim 6, wherein the connecting component is provided as at least two connecting buckles, wherein the connecting buckles are disposed on the front supporting portion and the side supporting portion respectively.

10. The posture correcting device as recited in claim 1, wherein an upper concave surface is further formed in the top surface of the front supporting portion, wherein the upper concave surface is adapted for the curve of the lower jaw of the user during wearing.

11. The posture correcting device as recited in claim 2, wherein an upper concave surface is further formed in the top surface of the front supporting portion, wherein the upper concave surface is adapted for the curve of the lower jaw of the user during wearing.

12. The posture correcting device as recited in claim 3, wherein an upper concave surface is further formed in the top surface of the front supporting portion, wherein the upper concave surface is adapted for the curve of the lower jaw of the user during wearing.

13. The posture correcting device as recited in claim 4, wherein an upper concave surface is further formed in the top surface of the front supporting portion, wherein the upper concave surface is adapted for the curve of the lower jaw of the user during wearing.

14. The posture correcting device as recited in claim 5, wherein an upper concave surface is further formed in the top surface of the front supporting portion, wherein the upper concave surface is adapted for the curve of the lower jaw of the user during wearing.

15. The posture correcting device as recited in claim 6, wherein an upper concave surface is further formed in the top surface of the front supporting portion, wherein the upper concave surface is adapted for the curve of the lower jaw of the user during wearing.

16. The posture correcting device as recited in claim 7, wherein an upper concave surface is further formed in the top surface of the front supporting portion, wherein the upper concave surface is adapted for the curve of the lower jaw of the user during wearing.

17. The posture correcting device as recited in claim 8, wherein an upper concave surface is further formed in the top surface of the front supporting portion, wherein the upper concave surface is adapted for the curve of the lower jaw of the user during wearing.

18. The posture correcting device as recited in claim 9, wherein an upper concave surface is further formed in the top surface of the front supporting portion, wherein the upper concave surface is adapted for the curve of the lower jaw of the user during wearing.
